(19) United States
(12) Patent Application Publication
(10) Pub. No.: US 2026/0120837 A1
(43) Pub. Date: Apr. 30, 2026

(54) METHODS OF DOSING THERAPEUTIC AGENTS

(71) Applicant: Fusion Pharmaceuticals Inc., Hamilton (CA)

(72) Inventors: Peng Guo, Lexington, MA (US); John Rhoden, Hingham, MA (US)

(21) Appl. No.: 18/968,741

(22) Filed: Dec. 4, 2024

Related U.S. Application Data
(63) Continuation of application No. PCT/US2023/067981, filed on Jun. 6, 2023.
(60) Provisional application No. 63/349,256, filed on Jun. 6, 2022.

(51) Int. Cl. G16H 20/10; G16B 15/30

(52) U.S. Cl. CPC G16H 20/10; G16B 15/30

(57) ABSTRACT

Disclosed are methods for dosing a drug for treating a medical condition in a subject in need thereof, comprising performing a first administering of the drug to the subject at various dose levels to provide a pharmacokinetic (PK) profile of the drug; obtaining parameters of the PK profile, wherein the parameters comprise Michaelis constant (K_{M}), maximum velocity (V_{M}), nonspecific first-order clearance (Cl), and maximal concentration (Cₘₐₓ); determining transitional concentration (Cₜᵣₐₙₛ) based on the parameters, wherein the PK profile of the drug exhibits a rapid transition at Cₜᵣₐₙₛ; formulating a therapeutic dose of the drug based on Cₜᵣₐₙₛ to reach a desired clinical endpoint at a pre-determined time point; and performing a second administering of the drug to the subject at the therapeutic dose for treating the medical condition.

### CROSS-REFERENCE TO RELATED APPLICATIONS

The application claims the benefit of, and priority to, U.S. Provisional Patent Application No. 63/349,256, filed on Jun. 6, 2022, the entire content of which is incorporated by reference herein.

### BACKGROUND

Transition zone, which is bound by linear phases in semi-log scale and where pharmacokinetic (PK) curve slopes change rapidly, is frequently observed in PK profiles of various therapeutic agents, whose PK profiles are of nonlinear nature. However, there is no quantitative way to precisely define the transition zone and its boundaries.

There is a need for methods of defining the transition zone of the PK profiles of therapeutic drugs, thereby determining therapeutic dosages of such drugs for treating medical conditions, such as cancer.

### SUMMARY

The present disclosure encompasses the insight that a key transitional concentration (Cₜᵣₐₙₛ) can be defined based on the parameters of the nonlinear pharmacokinetic (PK) model for a therapeutic agent (e.g., a drug). Mathematical analysis and numerical simulation are conducted to show that Cₜᵣₐₙₛ can be used to predict the transition zone of the PK profiles described by a few nonlinear PK models.

Typically, it is important to determine an “optimal” dose so that the drug concentration falls into the transition zone at a pre-determined time point to gain some therapeutic benefits. The well-defined Cₜᵣₐₙₛ as disclosed herein provides a quantitative approach to determine a therapeutic dose to achieve that goal.

Specifically, the present disclosure provides a method for dosing a drug for treating a medical condition (e.g., cancer) in a subject in need thereof, comprising performing a first administering of the drug to the subject at various dose levels to provide pharmacokinetic (PK) profiles of the drug; obtaining parameters of the PK model, wherein the parameters comprise Michaelis-Menten constant (K_{M}), maximum velocity (V_{M}), nonspecific first-order clearance (Cl), and maximal concentration (Cₘₐₓ); determining the transitional concentration (Cₜᵣₐₙₛ) based on the parameters, wherein the PK profile of the drug exhibits a rapid transition at Cₜᵣₐₙₛ; formulating a therapeutic dose (D_{T}) of the drug based on Cₜᵣₐₙₛ to reach a desired clinical endpoint; and performing a second administering of the drug to the subject at the Dr for treating the medical condition.

In some embodiments, the subject to be treated is a human.

In some embodiments, Cₜᵣₐₙₛ is determined according to formula sqrt(K_{M}×V_{M}/Cl).

In some embodiments, the PK profile is featured by one- or two-compartment PK models having either a nonlinear only clearance pathway or mixed (parallel) linear and nonlinear clearance pathways.

In some embodiments, the nonlinear clearance pathway features empirical Michaelis-Menten (M-M) equation.

In some embodiments, Cₜᵣₐₙₛ is greater than sqrt(⅓)×K_{M}.

In some embodiments, the nonlinear clearance pathway features target mediated drug disposition (TMDD).

In some embodiments, Cₜᵣₐₙₛ is determined according to formula sqrt(K_{M}×V_{M}/Cl), wherein K_{M}=K_{off}/Kₒₙ and V_{M}=Kᵢₙₜ×Rₜₒₜ×V_{c}, where Kₒₙ is the forward rate constant of drug-target binding with a unit of 1/concentration/time; K_{off} is the reverse rate constant of drug-target binding with a unit of 1/time; Kᵢₙₜ is the rate constant of internalization of drug-target complex with a unit of 1/time, Rₜₒₜ is the total target concentration, and Ve is the distribution volume of the central compartment.

In some embodiments, Cₜᵣₐₙₛ is determined according to formula sqrt(K_{M}×V_{M}/Cl), wherein K_{M}=(K_{off}+Kᵢₙₜ)/Kₒₙ and V_{M}=K_{syn}×V_{c}, where Kₒₙ is the forward rate constant of drug-target binding with a unit of 1/concentration/time; K_{off} is the reverse rate constant of drug-target binding with a unit of 1/time; Kᵢₙₜ is the rate constant of internalization of drug-target complex with a unit of 1/time, K_{syn} is the target synthesis rate constant, and V_{c} is the distribution volume of the central compartment.

In some embodiments, the desired clinical endpoint includes a time to reach Cₜᵣₐₙₛ, denoted as Tₜᵣₐₙₛ, that is determined according to the following equation where the PK is described by one compartment model with mixed linear and nonlinear clearance pathways:

$${T}_{trans} = \frac{{V}_{C}}{Cl} [ \frac{{K}_{M}}{{K}_{M} + \frac{{V}_{M}}{Cl}} ln ( \frac{{C}_{max}}{{C}_{trans}} ) + \frac{\frac{{V}_{M}}{Cl}}{{K}_{M} + \frac{{V}_{M}}{Cl}} ln ( \frac{{C}_{max} + \frac{{V}_{M}}{Cl} + {K}_{M}}{{C}_{trans} + \frac{{V}_{M}}{Cl} + {K}_{M}} ) ] .$$

When Cₘₐₓ is less than Cₜᵣₐₙₛ, Tₜᵣₐₙₛ is calculated to be negative and thereby undefined; when Cₘₐₓ is equal to Cₜᵣₐₙₛ, Tₜᵣₐₙₛ is calculated to be 0; when Cₘₐₓ is greater than Cₜᵣₐₙₛ, Tₜᵣₐₙₛ is calculated to be positive.

In some embodiments, the therapeutic dose (D_{T}) of the drug is formulated to reach Cₜᵣₐₙₛ at a pre-determined time point, wherein D_{T} is determined according to the following equation, where V_{c} is the distribution volume:

$${D}_{T} = {C}_{max} \times {V}_{c} .$$

In some embodiments, the desired clinical endpoint includes a time to reach Cₜᵣₐₙₛ, denoted as Tₜᵣₐₙₛ, that is determined according to the following equation where the PK is described by one compartment model with mixed linear and nonlinear clearance pathways:

$${T}_{trans} = \frac{{V}_{C}}{Cl} [ \frac{{K}_{M}}{{K}_{M} + \frac{{V}_{M}}{Cl}} ln ( \frac{{C}_{max}}{{C}_{trans}} ) + \frac{\frac{{V}_{M}}{Cl}}{{K}_{M} + \frac{{V}_{M}}{Cl}} ln ( \frac{{C}_{max} + \frac{{V}_{M}}{Cl} + {K}_{M}}{{C}_{trans} + \frac{{V}_{M}}{Cl} + {K}_{M}} ) ] ,$$

wherein Tₜᵣₐₙₛ is equal to the pre-determined time point.

In some embodiments, Tₜᵣₐₙₛ or the pre-determined time point is 14, 21, 28, 35, 42 days or longer.

In some embodiments, the therapeutic dose (D_{T}) of the drug is formulated to reach a plasma concentration (Cₚ) that is greater than Cₜᵣₐₙₛ for a pre-determined duration of time (T_{dur}), wherein D_{T} is determined according to the following equation, where V_{c} is the distribution volume:

$${D}_{T} = {C}_{max} \times {V}_{c} .$$

In some embodiments, the desired clinical endpoint includes a time to reach Cₜᵣₐₙₛ, denoted as Tₜᵣₐₙₛ, that is determined according to the following equation where the PK is described by one compartment model with mixed linear and nonlinear clearance pathways:

$${T}_{trans} = \frac{{V}_{C}}{Cl} [ \frac{{K}_{M}}{{K}_{M} + \frac{{V}_{M}}{Cl}} ln ( \frac{{C}_{max}}{{C}_{trans}} ) + \frac{\frac{{V}_{M}}{Cl}}{{K}_{M} + \frac{{V}_{M}}{Cl}} ln ( \frac{{C}_{max} + \frac{{V}_{M}}{Cl} + {K}_{M}}{{C}_{trans} + \frac{{V}_{M}}{Cl} + {K}_{M}} ) ] ,$$

wherein Tₜᵣₐₙₛ is greater than T_{dur}.

In some embodiments, the subject is a patient having a medical disorder (e.g., cancer).

In some embodiments, the drug is an antibody, a peptide, or a small molecule.

### BRIEF DESCRIPTION OF THE DRAWINGS

FIG. 1 illustrates dimensionless elimination rates for dimensionless number R being 4. Here the transitional concentration is calculated as sqrt(R)=2; at this transitional concentration, the total mass elimination rate is almost 1, relative to V_{M}, refer to black dot.

FIG. 2A illustrates dimensionless PK profile in a semi-log scale with dimensionless slopes at a few key concentrations for dimensionless concentration Xₘₐₓ being 100 and dimensionless number R being 10. FIG. 2B illustrates dimensionless slope vs dimensionless concentration. Both FIGS. 2A and 2B show that Cₜᵣₐₙₛ lies in the middle of the transition zone of the PK profile.

FIG. 3 illustrates dimensionless analytical solution and two approximate solutions and their convergence and divergence around the transition zone for dimensionless concentration Xₘₐₓ being 100 and dimensionless number R being 10.

FIG. 4 illustrates dimensionless PK profiles based on a PK model having two compartments with mixed (or parallel) linear and nonlinear clearance pathways for dimensionless concentration Xₘₐₓ being 100 and 1000, respectively; and dimensionless number R being 10; and dimensionless number Q/Cl being 4.0 and dimensionless number Vp/Vc being 2.5.

It is to be understood that the figures are not necessarily drawn to scale, nor are the objects in the figures necessarily drawn to scale in relationship to one another. The figures are depictions that are intended to bring clarity and understanding to various embodiments of systems and methods disclosed herein. Wherever possible, the same reference numbers will be used throughout the drawings to refer to the same or like parts. Moreover, it should be appreciated that the drawings are not intended to limit the scope of the present teachings in any way whatsoever.

### DETAILED DESCRIPTION

The present disclosure relates to methods of determining a dosage of a therapeutic agent (e.g., drug) for treating a medical condition (e.g., cancer).

Therapeutic agents like monoclonal antibodies (e.g., cetuximab) and some small molecules (e.g., phenytoin) exhibit nonlinear pharmacokinetics (PK) at a certain range of doses. The nonlinearity typically results from nonlinear kinetics in any step of key pharmacokinetic processes, such as absorption, distribution, metabolism, and elimination. Two major factors leading to nonlinear PK are the capacity-limited metabolism and target-mediated drug disposition (TMDD), see e.g., Jusko (1989), Ludden (1991), Levy (1994), and An (2017).

This disclosure provides a transitional concentration (Cₜᵣₐₙₛ), which is applicable for any therapeutic agent (e.g., drug) whose pharmacokinetics (PK) is described by nonlinear PK models comprising a few compartments (1, 2, or 3) and also including parallel linear and nonlinear clearance pathways; the nonlinear pathway includes but is not limited to TMDD and empirical Michaelis-Menten (M-M) equation. Typically, PK of such drugs exhibits rapid transitions at some concentrations; such transition can be captured by the defined Cₜᵣₐₙₛ.

It is known in the field that nonlinear PK are frequently described by compartmental models comprising mixed linear and nonlinear clearance pathways. The linear clearance pathway can be described by first order kinetics with a rate constant of Cl, which can be used to determine the mass clearance rate as Cl×Cp(t), where Cp(t) is the plasma concentration of the compound with respect to time. The nonlinear clearance pathway can be described by a TMDD model (whose governing equations are listed in Equations 7A/7B/7C below) or Michaelis-Menten equation, which has the following formula (1):

$$\frac{{V}_{M} \times Cp ( t )}{{K}_{M} + Cp ( t )} \qquad ( 1 )$$

where V_{M} (unit of mass/time) and K_{M} (unit of concentration) are M-M equation coefficients.

For PK models comprising mixed linear and nonlinear M-M clearance pathways, a transitional concentration, denoted herein as Cₜᵣₐₙₛ, is defined as:

$${C}_{trans} = sqrt ( {K}_{M} \times {V}_{M} / Cl ) \qquad ( 2 )$$

In Equation (2), sqrt(K_{M}×V_{M}/Cl) can also be expressed as

$$\sqrt{\frac{{K}_{M} \times {V}_{M}}{Cl}} or \sqrt{\frac{{V}_{M} \times {K}_{M}}{Cl}} ,$$

both of which are interchangeably used in this invention.

If only nonlinear M-M equation exits (i.e., Cl is zero), the transitional concentration is defined as:

$${C}_{trans} = {K}_{M} \qquad ( 3 )$$

Cₜᵣₐₙₛ is used to predict and locate the transition zone of the PK profiles. Determination of Cₜᵣₐₙₛ can be done in a few approaches: either directly calculated from the PK model parameters or estimated or visually determined from the observed PK profiles.

The definition of Cₜᵣₐₙₛ is demonstrated with one example; the PK of one drug is described by a one-compartment model with parallel linear (first order with rate constant Cl) and nonlinear clearance pathways; nonlinear pathway is described by M-M equation (with parameters of V_{M} and K_{M}). In this case, one dimensionless number can be defined as R=V_{M}/(Cl×K_{M}).

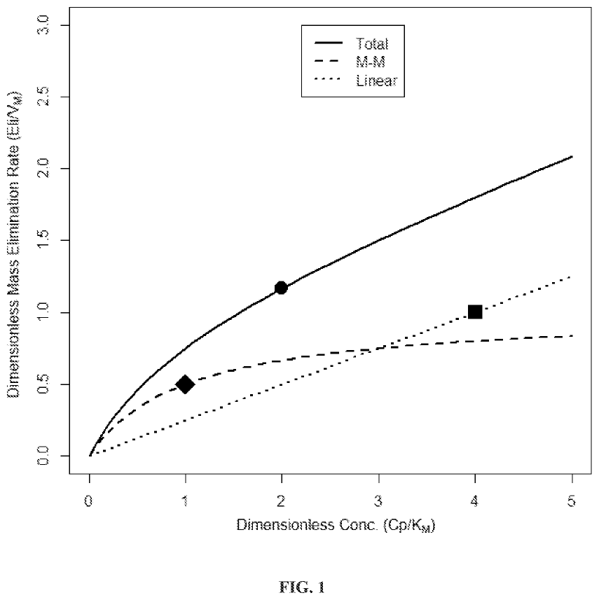

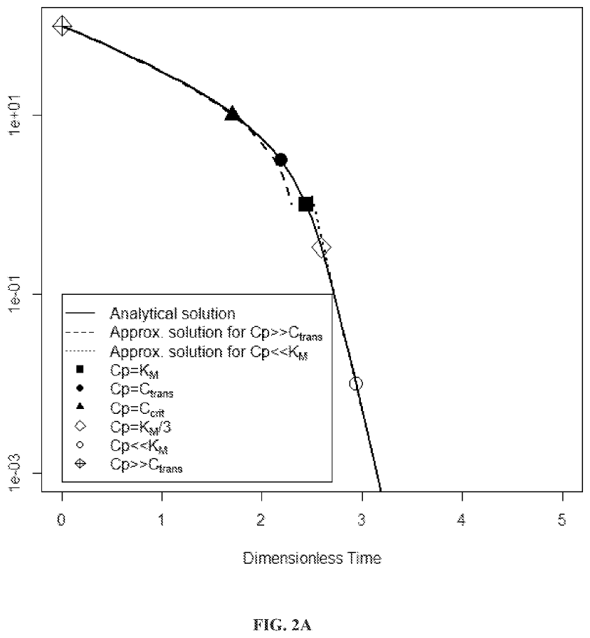

The transitional concentration Cₜᵣₐₙₛ is defined as follows. Factors to be considered include the mass elimination rates (i.e., Elimination in FIG. 1) of the first order clearance, the M-M equation, and the total rate (the sum of the first order clearance and the M-M equation). Cₜᵣₐₙₛ is defined as the concentration to let the total mass elimination rate equal to V_{M}. It is found that when R is greater than or equal to ⅓, dimensionless number sqrt(R) is the best choice for determining the transitional concentration, referring to FIG. 1; in dimensional form (relative to K_{M}), the transitional concentration is determined by formula K_{M}×sqrt(R), which is K_{M}×sqrt(V_{M}/(Cl×K_{M})), i.e., sqrt(K_{M}×V_{M}/Cl). When R is less than ⅓, drug PK profiles are almost linear no matter how much dose is administered via bolus injection as if the nonlinear clearance term described by M-M equation (1) is dropped.

In this description, two formulations are used: one in a dimensional form and the other in a dimensionless form. The latter has an advantage to reduce model parameters by at least 2 and to neatly summarize the results for various combinations of dimensional model parameters. In the dimensionless form, all concentrations are scaled relative to K_{M} while all slopes are scaled relative to Cl/Vc. Dimensionless number R, i.e., V_{M}/(Cl×K_{M}), can be referred to a critical concentration (C_{crit}); in dimensional form, C_{crit} is determined by formula R×K_{M}=V_{M}/Cl. The dimensionless transitional concentration is sqrt(R); in dimensional form, Cₜᵣₐₙₛ is determined by formula sqrt(K_{M}×V_{M}/Cl). The maximal concentration after bolus injection is Cₘₐₓ=D_{T}/Vc and its dimensionless form is denoted as Xₘₐₓ. which is determined by formula D_{T}/(V_{c}×K_{M}).

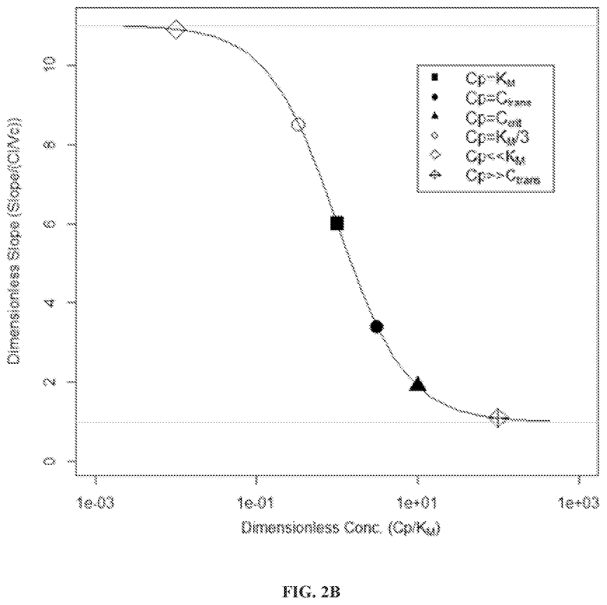

The use of Cₜᵣₐₙₛ to predict the transition zone is analyzed by two approaches. Firstly, considering the slope of the PK curve at a few key concentrations; it is found that the slope at Cₜᵣₐₙₛ lies in the middle part of the slopes of the PK curve, referring to FIG. 2, where drug PK is described by one compartment model with mixed linear and nonlinear clearance pathways.

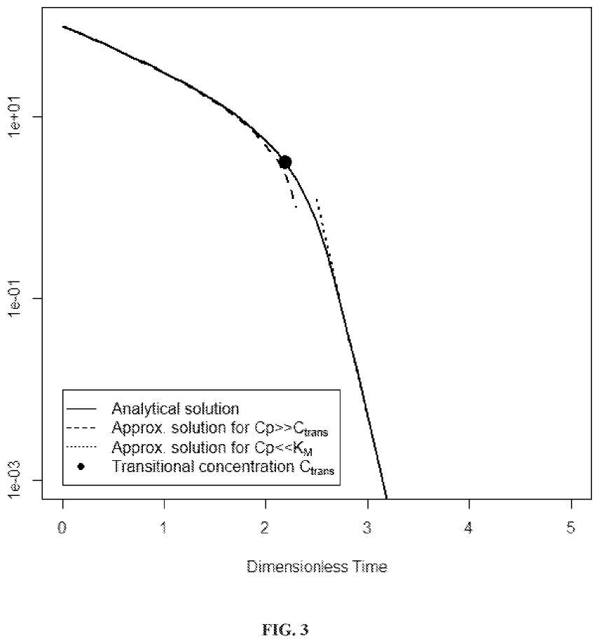

Secondly, two approximate solutions are obtained for two scenarios: (i) Cp far greater than Cₜᵣₐₙₛ, which is greater than K_{M}; and (ii) Cp far less than K_{M}. It is found that Cₜᵣₐₙₛ lies in the central region of the transition zone where those two approximate solutions diverge from the analytical solution at the boundaries of the transition zone. See FIG. 3, where drug PK is described by one compartment model with mixed linear and nonlinear clearance pathways.

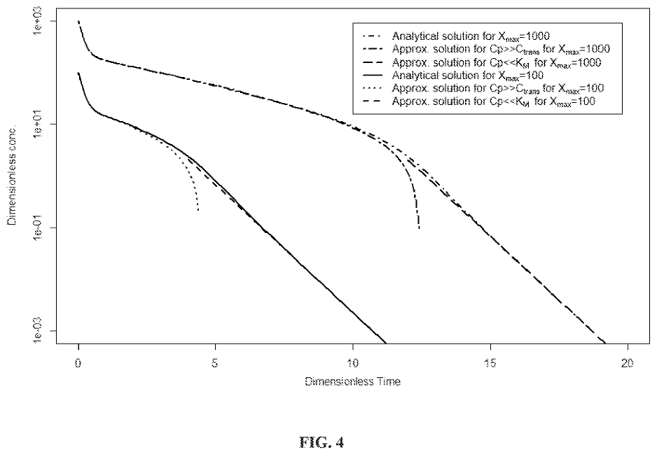

Further, it is found that the definition of Cₜᵣₐₙₛ is also applicable for PK models comprising two compartments with mixed (or parallel) linear and nonlinear (M-M) clearance pathways. See FIG. 4.

Referring to FIG. 4, Cₜᵣₐₙₛ can be defined in the same way as in the one compartment model shown in FIGS. 2-3, i.e., Cₜᵣₐₙₛ=sqrt(K_{M}×V_{M}/Cl); also, Cₜᵣₐₙₛ lies in the central region of the transition zone where two approximate solutions diverge from the analytical solution at the boundaries of the transition zone.

As mentioned before, there is currently no quantitative way to precisely define the transition zone of PK profiles of nonlinear nature.

As demonstrated in FIGS. 2-4 above, Cₜᵣₐₙₛ as disclosed herein can be used to predict the transition zone of PK profiles for various therapeutic agents such as antibodies and small molecule drugs. The existence of the transition zone in the PK profile is dependent on the therapeutic dose (Dose or D_{T}) and PK model parameters. For any therapeutic dose, Cₘₐₓ can be defined as:

$${C}_{max} = Dose / Vc \qquad ( 4 )$$

where Vc is the distribution volume of the central compartment where the linear and nonlinear clearance pathways are present and the bolus injection administration is occurred. If Cₘₐₓ is greater than Cₜᵣₐₙₛ, the time to reach Cₜᵣₐₙₛ, denoted herein as Tₜᵣₐₙₛ, is dependent on Cₘₐₓ and PK model parameters; if Cₘₐₓ is equal to Cₜᵣₐₙₛ, Tₜᵣₐₙₛ=0, i.e., the administration time; if Cₘₐₓ is less than Cₜᵣₐₙₛ, Tₜᵣₐₙₛ is not defined.

Generally, Tₜᵣₐₙₛ or the pre-determined time point can be any days between 1 day and 100 days (e.g., 1, 2, 3, 4, 5, 6, 7, 8, 9, 10, 20, 30, 40, 50, 60, 70, 80, 90 days; 1, 2, 3, 4, 5, 6, 7, 8, 9, 10 weeks, or 1, 2, 3 months). In some embodiments, Tₜᵣₐₙₛ or the pre-determined time point is 7, 14, 21, 28, 35, 42 days or longer. In certain embodiments, Tₜᵣₐₙₛ or the pre-determined time point is 14, 28, or 42 days.

For a PK model comprising only one compartment (whose distribution volume is Vc) and including mixed linear and nonlinear M-M clearance pathways, Tₜᵣₐₙₛ, can be defined by the following.

$${T}_{trans} = \frac{{V}_{C}}{Cl} [ \frac{{K}_{M}}{{K}_{M} + \frac{{V}_{M}}{Cl}} ln ( \frac{{C}_{max}}{{C}_{trans}} ) + \frac{\frac{{V}_{M}}{Cl}}{{K}_{M} + \frac{{V}_{M}}{Cl}} ln ( \frac{{C}_{max} + \frac{{V}_{M}}{Cl} + {K}_{M}}{{C}_{trans} + \frac{{V}_{M}}{Cl} + {K}_{M}} ) ] \qquad ( 5 )$$

where Vc is the distribution volume of the compartment, Cl is the clearance rate constant for the linear/first order clearance, K_{M} and V_{M} are M-M equation coefficients, Cₘₐₓ and Cₜᵣₐₙₛ are defined in Equations (4) and (2), respectively. With known values for PK model parameters (Vc, Cl, K_{M}, V_{M}) and Cₜᵣₐₙₛ defined, Tₜᵣₐₙₛ and Cₘₐₓ are related to each other according to Equation (5): if one is known, the other is uniquely determined. Equation (5) can be used to determine Cₘₐₓ and the therapeutical dose based on equation Dose=Cₘₐₓ×V_{c} for a given Tₜᵣₐₙₛ; or to determine Tₜᵣₐₙₛ with the known therapeutic dose and Cₘₐₓ.

Equation (5) is a special application at the transitional concentration Cₜᵣₐₙₛ of the analytical solution for the aforementioned PK model, where the analytical solution can be referenced to Wagner (1988) and Wagner (1993). The analytical solution was obtained for small molecule drugs before the advent of protein therapeutics (such as mAbs); it was used to calculate major PK parameters such as AUC (area under the curve) of drug products, the trough concentration, and accumulation ratio for multiple administrations. To our best knowledge, the analytical solution has not been used to predict dosage forms for protein therapeutics and/or monoclonal antibodies. This would be the first time to use the analytical solution for calculating the time to reach the transitional concentration Cₜᵣₐₙₛ, thereby formulating a therapeutic dose of the drug (e.g., antibody) based on Cₜᵣₐₙₛ to reach a desired clinical endpoint.

As disclosed herein, the term “desired clinical endpoint” refers to the effect of treating, ameliorating, or preventing a disease or a medical condition via pharmacological modulation of a target, receptor, or biochemical pathway in a target cell type or tissue of a subject (e.g., a human or a patient). For example, a desired clinical endpoint can be saturation of binding and internalization of therapeutic targets and modulation of the underlying intracellular biochemical signaling pathways to alleviate side effects or achieve a partial or complete remission in treating a disease (e.g., cancer).

For other PK models, such as a PK model comprising two compartments and including mixed linear and nonlinear M-M clearance pathways, Cₜᵣₐₙₛ is still defined by equation (2); Tₜᵣₐₙₛ can be obtained via numerically solving the governing equations for the PK model. The governing equations for such a model can be defined as below:

$$Vc \times \frac{dCp}{dt} = - Cl \times Cp - Q \times ( Cp - C 2 ) - {V}_{M} \times \frac{Cp}{Cp + {K}_{M}} \qquad ( 6 A )$$

$$Vp \times \frac{dC 2}{dt} = Q \times ( Cp - C 2 ) \qquad ( 6 B )$$

where Vc and Vp are distribution volumes of the central and peripheral compartments, Q is the exchange rate between two compartments, C2 is the drug concentration in the peripheral compartment defined as C2=Mp/Vp, where Mp is drug mass in the peripheral compartment. The initial conditions are:

$$Cp ( t = 0 ) = Dose / Vc = {C}_{max} and C 2 = 0 \qquad ( 6 C )$$

Equations 6A-6C shown above can be solved with any ordinary differential equation (ODE) solver such as Berkeley Madonna™, package deSolve of R, or various ODE solvers in MATLAB™. Once the numerical solution for Cp(t) is obtained, Turans, which is achieved when Cp(t) is equal to Cₜᵣₐₙₛ, can be read out and recorded for a given value of Cₘₐₓ/Dose.

In TMDD models, drug PK can be obtained via solving mechanistic equations describing the key processes of drug PK, including distribution, clearance, drug-target binding/unbinding and internalization. Assuming one TMDD model has one compartment with a distribution volume Vc and a first-order clearance rate Cl, the governing equations for such a TMDD model are defined as below:

$$\frac{dCp ( t )}{dt} = - {K}_{el} \times Cp ( t ) - {K}_{on} \times Cp ( t ) \times T + {K}_{off} \times CT \qquad ( 7 A )$$

$$\frac{dT}{dt} = {K}_{syn} - {K}_{deg} \times T - {K}_{on} \times Cp ( t ) \times T + {K}_{off} \times CT \qquad ( 7 B )$$

$$\frac{d ( CT )}{dt} = {K}_{on} \times Cp ( t ) \times T - {K}_{off} \times CT - {K}_{int} \times CT \qquad ( 7 C )$$

where Cp(t) is drug concentration in the compartment, T is free target concentration, and CT is the concentration of drug-target complex. Model parameters are defined as below:
Kₑₗ=Cl/Vc, the elimination rate constant with a unit of/time;
K_{syn}, the rate constant of target synthesis with a unit of concentration/time;
K_{deg}, the rate constant of degradation of free target with a unit of 1/time;
Kₒₙ, the forward rate constant of drug-target binding with a unit of 1/concentration/time;
K_{off}, the reverse rate constant of drug-target binding with a unit of 1/time;
Kᵢₙₜ, the rate constant of internalization of drug-target complex with a unit of 1/time.
   The initial conditions can be defined as below:

$$Cp ( t = 0 ) = Dose / Vc = {C}_{max} , T ( t = 0 ) = {K}_{syn} / {K}_{deg} , and CT ( t = 0 ) = 0 \qquad ( 7 D )$$

Equations 7A-7C with the initial conditions as defined in 7D can be solved with any ODE solver such as Berkeley Madonna™, package deSolve of R, or various ODE solvers in MATLAB™. Hence, their numerical solutions can be obtained accordingly. Once the numerical solution is obtained, Tₜᵣₐₙₛ to achieve Cams for any Cₘₐₓ value (where Cₘₐₓ is greater than Cₜᵣₐₙₛ) can be determined.

Under certain conditions such as quasi-equilibrium (QE) and quasi-steady state (QSS) conditions, a TMDD model is equivalent to a M-M model for a certain range of plasma concentrations. As a result, V_{M} and K_{M} of M-M equation can be expressed by the TMDD model parameters.

More specifically, under the QE condition, a TMDD model is equivalent to M-M model; see e.g., Yan et al., 2010; Mager & Krzyzanski, 2005. This equivalence is valid when the following conditions are satisfied:
1) Kᵢₙₜ=K_{deg} so that Tₜₒₜ=K_{syn}/K_{deg} is a constant (where Tₜₒₜ is total target concentration);
2) rapid binding, i.e, Kₒₙ×Cp(t)×T=K_{off}×CT is valid, for any time t;
3) Cp²≥(K_{d}×Tₜₒₜ) where K_{d} is the dissociation constant: K_{d}=K_{off}/Kₒₙ. (Yan et al., 2021).

Under the QE condition, V_{M} and K_{M} can be determined as below:

$${V}_{M} = {K}_{int} \times {T}_{tot} \times Vc \qquad ( 8 )$$

$${K}_{M} = {K}_{d} = {K}_{off} / {K}_{on} \qquad ( 9 )$$

where Cₜᵣₐₙₛ is also defined by Equation (2) with V_{M} and K_{M} defined in Equation (8) and (9). Expressed in an explicit form by TMDD model parameters, Cₜᵣₐₙₛ is determined as below:

$${C}_{trans} = \sqrt{\frac{{V}_{M} \times {K}_{M}}{Cl}} = \sqrt{\frac{{K}_{int} \times {T}_{tot} \times {V}_{C}}{Cl} \times \frac{{K}_{off}}{{K}_{on}}} \qquad ( 10 )$$

Alternatively, under the QSS condition, a TMDD model is also equivalent to M-M model; see e.g., Gibiansky et al., 2008. The equivalence is valid for rapid binding and for Cp²≥(K_{QSS}×Tₜₒₜ) where K_{QSS}=(K_{off}+Kᵢₙₜ)/Kₒₙ and Tₜₒₜ is a constant. It is demonstrated that QSS approximation is preferred over QE when Kim is not negligible compared to K_{off}.

Under the QSS condition, V_{M} and K_{M} can be determined as below:

$${V}_{M} = {K}_{syn} \times Vc \qquad ( 11 )$$

$${K}_{M} = {K}_{QSS} = ( {K}_{off} + {K}_{int} ) / {K}_{on} \qquad ( 12 )$$

where Cₜᵣₐₙₛ is also defined by Equation (2) with V_{M} and K_{M} defined in Equation (11) and (12). Expressed in an explicit form by TMDD model parameters, Cₜᵣₐₙₛ is determined as below:

$${C}_{trans} = \sqrt{\frac{{V}_{M} \times {K}_{M}}{c 1}} = \sqrt{\frac{{K}_{syn} \times {V}_{C}}{c 1} \times \frac{{K}_{off} + {K}_{int}}{{K}_{on}}} \qquad ( 13 )$$

For typical TMDD model described by Equations 7A-7D, its analytical solution is generally hard to be obtained while its numerical solution can be obtained with any ODE solver. Once its numerical solution is obtained, Tₜᵣₐₙₛ to achieve Cₜᵣₐₙₛ for any Cₘₐₓ value (where Cₘₐₓ is greater than Cₜᵣₐₙₛ) can be found.

The method disclosed herein can be used to determine the therapeutic dose for any given Turans with known PK model parameters. The following examples are merely illustrative for demonstrating how Cams is determined and how it is used to determine the therapeutic dosage of a drug to reach a desired clinical endpoint.

### Examples

### Example 1: Determination of Cₜᵣₐₙₛ of an Antibody Whose PK in Humans is Described by a Two-Compartment Model with Nonlinear Only Clearance Pathway

This example demonstrates how Cₜᵣₐₙₛ is determined for two antibodies: cetuximab and alemtuzumab, where their PK are described by a two-compartment model with nonlinear only clearance pathway described by M-M equation. For this kind of PK model, Cₜᵣₐₙₛ is determined according to Equation (3):

$${C}_{trans} = {K}_{M} \qquad ( 3 )$$

TABLE 1
PK parameters and calculated
Cₜᵣₐₙₛ for cetuximab and alemtuzumab

| Drug name | Cetuximab | Alemtuzumab |
|---|---|---|
| PK Model | Two-Compartment Model with nonlinear M-M clearance | |
| Vc | 2.83 (L) | 11.3 (L) |
| Vp | 2.43 (L) | 41.5 (L) |
| Cl | NA | NA |
| Q | 0.103 (L/hour) | 1.05 (L/hour) |
| V_{M} | 4.38 (mg/hour) | 1020 (μg/hour) |
| K_{M} | 74 (μg/mL) | 338 (μg/L) |
| Cₜᵣₐₙₛ | 74 (μg/mL) | 338 (μg/L) |

PK parameters listed in Table 1, i.e., Vc, Vp, Q, V_{M} and K_{M}, were obtained based on the protocols disclosed in Dirks et al., 2008 and Mould et al., 2007, with modification to be consistent with the current model description provided herein.

### Example 2: Determination of Cₜᵣₐₙₛ of a Small Molecule Drug Whose PK in Rats is Described by a Two-Compartment Model with Nonlinear Only Clearance Pathway

This example demonstrates how Cₜᵣₐₙₛ is determined for small molecule phenytoin. Its PK in rats is described by a two-compartment model with nonlinear only clearance described by M-M equation. For this kind of PK model, Cₜᵣₐₙₛ is determined according to Equation (3):

$${C}_{trans} = {K}_{M} \qquad ( 3 )$$

TABLE 2
PK parameters for rats and calculated Cₜᵣₐₙₛ for phenytoin

| Drug name | Phenytoin |
|---|---|
| PK Model | Two-Compartment Model with nonlinear only |
| | clearance pathway |
| Vc (mL) | 406 |
| Cl (mL/min) | NA |
| K₁₂ (/min) | 0.197 |
| K₂₁ (/min) | 0.060 |
| Q (mL/min) = K₁₂ × Vc | 79.98 |
| Vp (mL) = Q/K₂₁ | 1333 |
| V_{M} (μg/min) | 272 |
| K_{M} (μg/mL) | 5.9 |
| Cₜᵣₐₙₛ (μg/mL) | 5.9 |

PK parameters listed in Table 2, i.e., Vc, Vp, K₁₂, K₂₁, Q, V_{M} and K_{M}, were obtained based on the protocols disclosed in Della Paschoa et al., 1998, with modification to be consistent with the current model description provided herein.

### Example 3: Determination of Cₜᵣₐₙₛ of an Antibody Whose PK is Described by a Two-Compartment Model with Mixed Linear and Nonlinear Clearance Pathways (M-M Equation)

This example demonstrates how Cₜᵣₐₙₛ is determined for four antibodies: tocilizumab, sibrotuzumab, panitumumab, and vedolizumab, where their PKs are described by a two-compartment model with mixed linear and nonlinear clearance pathways (described by M-M equation). For this kind of PK model, Cₜᵣₐₙₛ is determined according to Equation (2):

$${C}_{trans} = sqrt ( {K}_{M} \times {V}_{M} / C 1 ) \qquad ( 2 )$$

TABLE 3
PK parameters and calculated Cₜᵣₐₙₛ for four antibodies

| Drug | Tocilizumab | Sibrotuzumab | Panitumumab | Vedolizumab |
|---|---|---|---|---|
| name | | | | |
| Model | Two-Compartment Model with mixed linear | | | |
| | and nonlinear M-M clearance pathways | | | |
| Vc (L) | 3.5 | 4.13 | 3.66 | 3.16 |
| Vp (L) | 2.9 | 3.19 | 2.58 | 1.84 |
| Cl (L/day) | 0.3 | 0.530 | 0.269 | 1.10 |
| Q (L/day) | 0.2 | 0.902 | 0.389 | 0.161 |
| V_{M} | 7.5 | 0.811 | 10.6 | 0.238 |
| (mg/day) | | | | |
| K_{M} | 2.7 | 0.219 | 0.401 | 0.851 |
| (μg/mL) | | | | |
| Cₜᵣₐₙₛ | 8.22 | 0.579 | 3.98 | 0.429 |
| (μg/mL) | | | | |

PK parameters listed in Table 3, i.e., Vc, Cl, Vp, Q, V_{M} and K_{M}, were obtained based on the protocols disclosed in Frey et al., 2010; Kloft et al., 2004; Ma et al., 2009; and Okamoto et al., 2021, with modification to be consistent with the current model description provided here.

### Example 4: Determination of Cₜᵣₐₙₛ of an Antibody Whose PK in Humans and/or Monkeys is Described by TMDD Model Under the Quasi-Equilibrium (QE) Condition

This example demonstrates how Cₜᵣₐₙₛ is determined for two antibodies: TRX1 and mAb-7, where their PKs are described by a two-compartment model with TMDD model under rapid binding and QE conditions. For this kind of PK model, Cₜᵣₐₙₛ is determined according to Equation (2), specified by Equation (10):

$${C}_{trans} = sqrt ( {K}_{M} \times {V}_{M} / C 1 ) \qquad ( 2 )$$

$${C}_{trans} = \sqrt{\frac{{V}_{M} \times {K}_{M}}{c 1}} = \sqrt{\frac{{K}_{int} \times {T}_{tot} \times {V}_{C}}{c 1} \times \frac{{K}_{off}}{{K}_{on}}} \qquad ( 10 )$$

where V_{M} and K_{M} are calculated from TMDD model parameters according to Equations (8) and (9):

$${V}_{M} = {K}_{int} \times {T}_{tot} \times {V}_{C} \qquad ( 8 )$$

$${K}_{M} = {K}_{d} = {K}_{off} / {K}_{on} \qquad ( 9 )$$

TABLE 4
PK (TMDD) model parameters and calculated Cₜᵣₐₙₛ for two antibodies

| Drug name | TRX1 for a human (70 kg) | mAb-7 for Monkey |
|---|---|---|
| Model | Two-Compartment model with | |
| | TMDD model (QE approximation) | |
| Kₑₗ (/day) | 0.078 | 0.14 |
| Vc (L) | 2.92* | 0.132 |
| K_{cp} = Q/Vc | 0.649 | 1.25 |
| K_{pc}= Q/Vp | 0.874 | 0.941 |
| Kₒₙ (/nM/day) | 0.753 | NA |
| K_{off} (/day) | 14.6 | NA |
| Kᵢₙₜ (/day) | 3.93 | 5.07 |
| K_{syn} (/day) | 38.1 | 18.14 |
| K_{deg} (/day) | 0.694 | 15.5 |
| Tₜₒₜ (nM) | 54.9^{#} | 1.17 |
| K_{d} = K_{off}/Kₒₙ (nM) | 19.39 | 9.37 |
| Cl = Kₑₗ × Vc (L/day) | 0.228 | 0.0185 |
| V_{M} = Kᵢₙₜ × Tₜₒₜ × Vc (nmol/day) | 629.8 | 0.783 |
| K_{M} = K_{d} (nM) | 19.38 | 9.37 |
| Cₜᵣₐₙₛ (nM) | 231.6 | 19.91 |

*Assuming a human subject of 70 kg weight.
^{#}From Yan et al., 2010.

PK parameters listed in Table 4 were obtained based on the protocols disclosed in Ng et al., 2006, Yan et al., 2010, and Singh et al., 2015, with modification to be consistent with the model description listed in Equations (7A)-(7D).

### Example 5: Determination of Cₜᵣₐₙₛ of an Antibody Whose PK is Described by TMDD Model Under the Quasi-Steady-State (QSS) Condition

This example demonstrates how Cₜᵣₐₙₛ is determined for two antibodies: mavrilimumab and efalizumab, where their PKs in humans are described by a two-compartment model with TMDD model under the QSS condition. For this kind of PK model, Cₜᵣₐₙₛ is determined according to Equation (2), specified by Equation (13).

$${C}_{trans} = sqrt ( {K}_{M} \times {V}_{M} / C 1 ) \qquad ( 2 )$$

$${C}_{trans} = \sqrt{\frac{{V}_{M} \times {K}_{M}}{c 1}} = \sqrt{\frac{{K}_{syn} \times {V}_{C}}{c 1} \times \frac{{K}_{off} + {K}_{int}}{{K}_{on}}} \qquad ( 13 )$$

where V_{M} and K_{M} are calculated from TMDD model parameters according to Equations (11) and (12).

$${V}_{M} = {K}_{syn} \times {V}_{C} \qquad ( 11 )$$

$${K}_{M} = {K}_{QSS} = {X}_{off} + {K}_{int} ) / {K}_{on} \qquad ( 12 )$$

TABLE 5
PK (TMDD) model parameters and calculated
Cₜᵣₐₙₛ for two antibodies

| Drug name | Mavrilimumab | Nimotuzumab |
|---|---|---|
| Model | Two-Compartment model with | |
| | TMDD model (QSS approximation) | |
| Vc (L) | 2.8 | 1.43 |
| Vp (L) | 5.6 | 18.5 |
| Cl | 0.3 (L/day) | 0.000703 (L/hour) |
| Q | 1.7 (L/day) | 0.00322 (L/hour) |
| Kₒₙ | 11 (/nM/day) | NA |
| K_{off} | 10 (/day) | NA |
| K_{syn} | 2.4 (nM/day) | 1.43 (μg/mL/hour) |
| K_{deg} | 2.2 (/day) | 5.5 (/hour) |
| Kᵢₙₜ | 2.2 (/day) | 0.148 (/hour) |
| Tₜₒₜ = K_{syn}/K_{deg} | 1.09 (nM) | 0.26 (μg/mL) |
| K_{QSS} = (Kᵢₙₜ + K_{off})/Kₒₙ | 1.1 (nM) | 6.96 (mg/L) |
| V_{M} = K_{syn}Vc | 6.72 (nmol/Day) | 2.045 (mg/hour) |
| K_{M} = K_{QSS} | 1.1 (nM) | 6.96 (mg/L) |
| Cₜᵣₐₙₛ | 4.96 (nM) | 142.3 (μg/mL) |

PK parameters listed in Table S were obtained based on the protocols disclosed in Stein & Peletier, 2018 and Rodriguez-Vera et al., 2015, with modification to be consistent with the current model description listed in Equations (7A)-(7D).

### Example 6: Determination of a Therapeutic Dose of One Compound to Reach Cₜᵣₐₙₛ at a Certain Time Point

This example demonstrates how Cₜᵣₐₙₛ is used to determine a therapeutic dose of evolocumab in humans, where its PK is described by a one-compartment model with mixed linear and nonlinear clearance pathways. Tₜᵣₐₙₛ to reach the transitional concentration Cₜᵣₐₙₛ for any dose level Cₘₐₓ is determined according to Equation(S). When Tₜᵣₐₙₛ and Cₜᵣₐₙₛ are known, Cₘₐₓ and the therapeutic dose can be determined as follows.

Evolocumab PK model parameters such as Vc, Cl, K_{M} and V_{M} are provided in Table 6 below. According to Equation (2), Cₜᵣₐₙₛ is determined to be 115.18 nM or 17.28 mg/L assuming the molecular weight of 150 kDa or 150 kg/mol for a typical antibody. Tₜᵣₐₙₛ is pre-determined to be 28 days. Tₜᵣₐₙₛ and Cₜᵣₐₙₛ can be used to determine Cₘₐₓ based on Equation(S). Cₘₐₓ can be obtained from Equation (5) via any root solver as provided in MATLAB™ and RStudio.

With R (version x64 4.0.5), Cₘₐₓ is determined to be 536.4 nM. which is converted into 80.46 mg/L as shown below assuming the molecular weight of 150 kDa or 150 kg/mol for a typical antibody.
Cₘₐₓ=536.4 nM, which is equivalent to 536.4 nmol/L×150,000 g/mol, which is equivalent to 536.4×0.15 mg/L, i.e., 80.46 mg/L
   The therapeutic dose (Dose or Dr) is calculated as shown below:

$${D}_{T} = {C}_{max} \times Vc = 80.46 mg / L \times 5.18 L = 416.8 mg .$$

Hence, the desired D_{T} is 416.8 mg to reach Cₜᵣₐₙₛ at Tₜᵣₐₙₛ=28 day.

TABLE 6
PK model parameters and calculated Cₜᵣₐₙₛ for Evolocumab

| Drug name | Evolocumab |
|---|---|
| Model | One-Comp, Nonlinear MM |
| Vc (L) | 5.18 |
| Cl (L/day) | 0.105 |
| V_{M} (nmol/day) | 51.02 |
| K_{M} (nM) | 27.3 |
| Cₜᵣₐₙₛ (nM) | 115.18 |
| Cₜᵣₐₙₛ (mg/L) | 17.28 |

PK parameters listed in Table 6 were obtained based on the protocols disclosed in Kuchimanchi et al., 2018, with modification to be consistent with the current model description listed in Equations (2) and (4).

### Example 7: Determination of the Minimal Dose Level to have Plasma Concentration Cp(t) Above Cₜᵣₐₙₛ for a Certain Time Duration

This example demonstrates how Cₜᵣₐₙₛ is used to determine a therapeutic dose of tocilizumab, where its PK in humans is described by a two-compartment model with mixed linear and nonlinear pathways.

This example requires that Tₜᵣₐₙₛ for the desired therapeutic dose be greater than the pre-determined duration time point post-administration, which is denoted herein as T_{dur}.

PK model parameters for tocilizumab such as Vc, Cl, Vp, Q, K_{M} and V_{M} are provided in Example 3. Cₜᵣₐₙₛ is determined to be 8.22 μg/mL or 8.22 mg/L; Tur is pre-determined as 28 days, which is equal to or less than Tₜᵣₐₙₛ. Cₘₐₓ for Tₜᵣₐₙₛ=28 day can be obtained via solving equations (6A/6B/6C) via any ODE solver. With package deSolve of R (version x64 4.0.5), Cₘₐₓ is determined to be 237.2 mg/L.

The therapeutic dose (D_{T}) is calculated as shown below:

$${D}_{T} = {C}_{max} \times V c = 237.2 mg / L \times 3.5 L = 830.2 mg$$

It is found that Tₜᵣₐₙₛ correlates with Cₘₐₓ as the greater the Cₘₐₓ, the greater the Tₜᵣₐₙₛ. Having Tₜᵣₐₙₛ greater than T_{dur}, the minimal Cₘₐₓ value is 237.2 mg/L; and the minimal dose to have tocilizumab reach a Cp(t) above Cₜᵣₐₙₛ for at least 28 days is 830.2 mg.

### REFERENCES

1. W J Jusko. Pharmacokinetics of capacity-limited systems. J Clin Pharmacol. 29:488-493, 1989.
2. T M Ludden. Nonlinear pharmacokinetics. Clinical Pharmacokinetics. 20:429-446, 1991.
3. G Levy. Pharmacologic target-mediated drug disposition. Clin Pharmacol Ther 56:248-252, 1994.
4. G H An. Small-molecule compounds exhibiting target-mediated drug disposition (TMDD): a minireview. The Journal of Clinical Pharmacology 57:137-150, 2017.
5. John G. Wagner. Direct Method of Simulating Concentration-Time Data for Michaelis-Menten Elimination. Drug Intelligence & Clinical Pharmacy, 22:381-386, 1988.
6. John G. Wagner. 1993. Pharmacokinetics for the pharmaceutical scientist. Technomic Publishing Company, Inc. Lancaster.
7. X Y Yan, D E Mager and W Krzyzanski. Selection between Michaelis-Menten and target-mediated drug disposition pharmacokinetic models. J Pharmacokinet Pharmacodyn. 37:25-47, 2010.
8. Mager, D. E. & Krzyzanski, W. Quasi-equilibrium pharmacokinetic model for drugs exhibiting target-mediated drug disposition. Pharm. Res. 22, 1589-1596 (2005). doi: 10.1007/s11095-005-6650-0.
9. Gibiansky, L., Gibiansky, E., Kakkar, T. & Ma, P. Approximations of the target mediated drug disposition model and identifiability of model parameters. J. Pharmacokinet Pharmacodyn. 35, 573-591 (2008). doi: 10.1007/s10928-008-9102-8.
10. Dirks N L, Nolting A, Kovar A, Meibohm B. Population pharmacokinetics of cetuximab in patients with squamous cell carcinoma of the head and neck. J Clin Pharmacol. 2008; 48 (3): 267-78.
11. Mould D R, Baumann A, Kuhlmann J, Keating M J, Weitman S, Hillmen P, et al. Population pharmacokinetics-pharmacodynamics of alemtuzumab (Campath) in patients with chronic lymphocytic leukaemia and its link to treatment response. Br J Clin Pharmacol. 2007; 64 (3): 278-91.
12. O E Della Paschoa, J W Mandema, R A Voskuyl and M Danhof. Pharmacokinetic-Pharmacodynamic Modeling of the Anticonvulsant and Electroencephalogram Effects of Phenytoin in Rats. Journal of Pharmacology and Experimental Therapeutics February 1998, 284 (2) 460-466.
13. Frey N, Grange S, Woodworth T. Population pharmacokinetic analysis of tocilizumab in patients with rheumatoid arthritis. J Clin Pharmacol. 2010; 50 (7): 754-66.
14. C. Kloft, E-U. Graefe, P. Tanswell, A. M. Scott, R. Hofheinz, A. Amelsberg & M. O. Karlsson. Population Pharmacokinetics of Sibrotuzumab, A Novel Therapeutic Monoclonal Antibody, in Cancer Patients. Investigational New Drugs 22:39-52, 2004.
15. Ma P, Yang B B, Wang Y M, Peterson M, Narayanan A, Sutjandra L, et al. Population pharmacokinetic analysis of panitumumab in patients with advanced solid tumors. J Clin Pharmacol. 2009: 49 (10): 1142-56.
16. H Okamoto, N L Dirks, M Rosario, T Hori, T Hibi. Population pharmacokinetics of vedolizumab in Asian and non-Asian patients with ulcerative colitis and Crohn's disease. Intest Res 2021: 19 (1): 95-105.
17. Ng C M, Stefanich E, Anand B S, Fielder P J, Vaickus L. Pharmacokinetics/pharmacodynamics of nondepleting anti-CD4 monoclonal antibody (TRX1) in healthy human volunteers. Pharm Res. 2006; 23 (1): 95-103.
18. Aman P. Singh, Wojciech Krzyzanski, Steven W. Martin, Gregory Weber, Alison Betts, Alaa Ahmad, Anson Abraham, Anup Zutshi, John Lin, & Pratap Singh. Quantitative Prediction of Human Pharmacokinetics for mAbs Exhibiting Target-Mediated Disposition. AAPSJ, 2015; 17:389-399.
19. A M Stein & L A Peletier. Predicting the onset of nonlinear pharmacokinetics. CPT: Pharmacometrics Syst. Pharmacol. 2018. 7:670-677.
20. Leyanis Rodríguez-Vera, Mayra Ramos-Suzarte, Eduardo Fernández-Sánchez, Jorge Luis Soriano, Concepción Peraire Guitart, Gilberto Castañeda Hernández, Carlos O. Jacobo-Cabral, Niurys de Castro Suárez. Semimechanistic model to characterize nonlinear pharmacokinetics of nimotuzumab in patients with advanced breast cancer. J Clinical Pharmacology. 55:888-898, 2015.
21. Mita Kuchimanchi, Anita Grover, Maurice G. Emery, Ransi Somaratne, Scott M. Wasserman, John P. Gibbs & Sameer Doshi. Population pharmacokinetics and exposure-response modeling and simulation for evolocumab in healthy volunteers and patients with hypercholesterolemia. Journal of Pharmacokinetics and Pharmacodynamics 45:505-522, 2018.

### OTHER EMBODIMENTS

Those skilled in the art will recognize or be able to ascertain using no more than routine experimentation, many equivalents to the specific embodiments described herein. Such equivalents are intended to be encompassed by the following claims.

1. A method for dosing a drug for treating a medical condition in a subject in need thereof, comprising:
performing a first administering of the drug to the subject at various dose levels to provide a pharmacokinetic (PK) profile of the drug;
obtaining parameters of the PK profile, wherein the parameters comprise Michaelis-Menten constant (K_{M}), maximum velocity (V_{M}), nonspecific first-order clearance (Cl), and maximal concentration (Cₘₐₓ);
determining transitional concentration (Cₜᵣₐₙₛ) based on the parameters, wherein the PK profile of the drug exhibits a rapid transition at Cₜᵣₐₙₛ;
formulating a therapeutic dose (D_{T}) of the drug based on Cₜᵣₐₙₛ to reach a desired clinical endpoint at a pre-determined time point; and
performing a second administering of the drug to the subject at the D_{T} for treating the medical condition.

2. The method of claim 1, where the subject is a human.

3. The method of claim 1, wherein Cₜᵣₐₙₛ is determined according to formula sqrt(K_{M}×V_{M}/Cl).

4. The method of claim 1, wherein the PK profile is featured by one- or two-compartment PK models having either a nonlinear only clearance pathway or mixed linear and nonlinear clearance pathways.

5. The method of claim 4, wherein the nonlinear clearance pathway features empirical Michaelis-Menten (M-M) equation.

6. The method of claim 1, where Cₜᵣₐₙₛ is greater than sqrt(⅓)×K_{M}.

7. The method of claim 4, wherein the nonlinear clearance pathway features target mediated drug disposition (TMDD).

8. The method of claim 7, wherein Cₜᵣₐₙₛ is determined according to formula sqrt(K_{M}×V_{M}/Cl), wherein K_{M}=K_{off}/Kₒₙ and V_{M}=Kᵢₙₜ×Tₜₒₜ×V_{c}, where Kₒₙ is the forward rate constant of drug-target binding with a unit of 1/concentration/time; K_{off} is the reverse rate constant of drug-target binding with a unit of 1/time; Kᵢₙₜ is the rate constant of internalization of drug-target complex with a unit of 1/time, Tₜₒₜ is the total target concentration, and V_{c} is the distribution volume of a central compartment.

9. The method of claim 7, wherein Cₜᵣₐₙₛ is determined according to formula sqrt(K_{M}×V_{M}/Cl), wherein K_{M}=(K_{off}+Kᵢₙₜ)/Kₒₙ and V_{M}=K_{syn}×V_{c}, where Kₒₙ is the forward rate constant of drug-target binding with a unit of 1/concentration/time; K_{off} is the reverse rate constant of drug-target binding with a unit of 1/time; Kᵢₙₜ is the rate constant of internalization of drug-target complex with a unit of 1/time, K_{syn} is the target synthesis rate constant, and V_{c} is the distribution volume of a central compartment.

10. The method of claim 1, wherein the desired clinical endpoint includes a time to reach Cₜᵣₐₙₛ, denoted as Tₜᵣₐₙₛ, that is determined according to the following equation:
$${T}_{trans} = \frac{{V}_{C}}{C 1} [ \frac{{K}_{M}}{{K}_{M} + \frac{{V}_{M}}{C 1}} ln ( \frac{{C}_{max}}{{C}_{trans}} ) + \frac{\frac{{V}_{M}}{C 1}}{{K}_{M} + \frac{{V}_{M}}{C 1}} ln ( \frac{{C}_{max} + \frac{{V}_{M}}{C 1} + {K}_{M}}{{C}_{trans} + \frac{{V}_{M}}{C 1} + {K}_{M}} ) ]$$

11. The method of claim 1, wherein the therapeutic dose (D_{T}) of the drug is formulated to reach Cₜᵣₐₙₛ at a pre-determined time point, wherein D_{T} is determined according to the following equation, where V is the distribution volume:
$${D}_{T} = {C}_{max} \times {V}_{c} .$$

12. The method of claim 11, wherein the desired clinical endpoint includes a time to reach Cₜᵣₐₙₛ, denoted as Tₜᵣₐₙₛ, that is determined according to the following equation
$${T}_{trans} = \frac{{V}_{C}}{C 1} [ \frac{{K}_{M}}{{K}_{M} + \frac{{V}_{M}}{C 1}} ln ( \frac{{C}_{max}}{{C}_{trans}} ) + \frac{\frac{{V}_{M}}{C 1}}{{K}_{M} + \frac{{V}_{M}}{C 1}} ln ( \frac{{C}_{max} + \frac{{V}_{M}}{C 1} + {K}_{M}}{{C}_{trans} + \frac{{V}_{M}}{C 1} + {K}_{M}} ) ] ,$$
wherein Tₜᵣₐₙₛ is equal to the pre-determined time point.

13. The method of claim 11, wherein Tₜᵣₐₙₛ or the pre-determined time point is 28 days or longer.

14. The method of claim 1, wherein the therapeutic dose (D_{T}) of the drug is formulated to reach a plasma concentration (Cₚ) that is greater than Cₜᵣₐₙₛ for a pre-determined duration of time (T_{dur}), wherein D_{T} is determined according to the following equation, where V_{c} is the distribution volume:
$${D}_{T} = {C}_{max} \times {V}_{c} .$$

15. The method of claim 14, wherein the desired clinical endpoint includes a time to reach Cₜᵣₐₙₛ, denoted as Tₜᵣₐₙₛ, that is determined according to the following equation:
$${T}_{trans} = \frac{{V}_{C}}{C 1} [ \frac{{K}_{M}}{{K}_{M} + \frac{{V}_{M}}{C 1}} ln ( \frac{{C}_{max}}{{C}_{trans}} ) + \frac{\frac{{V}_{M}}{C 1}}{{K}_{M} + \frac{{V}_{M}}{C 1}} ln ( \frac{{C}_{max} + \frac{{V}_{M}}{C 1} + {K}_{M}}{{C}_{trans} + \frac{{V}_{M}}{C 1} + {K}_{M}} ) ] ,$$
wherein Tₜᵣₐₙₛ is greater than T_{dur}.

16. The method of claim 1, where the subject is a patient having a medical disorder.

17. The method of claim 16, where the medical disorder is cancer.

18. The method of claim 1, where the drug is an antibody, a peptide, or a small molecule.
